(19) United States
(12) Patent Application Publication
(10) Pub. No.: US 2025/0268485 A1
(43) Pub. Date: Aug. 28, 2025

(54) MAGNETIC RESONANCE LOCAL COIL ARRANGEMENT IN MULTIPLE PARTS WITH A LOWHEIGHT BOTTOM SECTION

(71) Applicant: Siemens Healthineers AG, Forchheim (DE)

(72) Inventor: Stephan Biber, Erlangen (DE)

(21) Appl. No.: 19/063,670

(22) Filed: Feb. 26, 2025

(30) Foreign Application Priority Data
Feb. 26, 2024 (DE) 10 2024 201 746.9

(51) Int. Cl. A61B 5/055; A61B 5/00; G01R 33/34

(52) U.S. Cl. CPC A61B 5/055; A61B 5/704; G01R 33/34084; A61B 2562/227

(57) ABSTRACT

A magnetic resonance local coil arrangement, a system with a magnetic resonance local coil arrangement and patient support apparatus, a magnetic resonance apparatus, and a use of such apparatuses are provided. The magnetic resonance local coil arrangement includes a bottom section and a top section. In a connected state, the bottom section and the top section delimit a spatial volume in which a body part of a patient may be supported during a magnetic resonance examination. The bottom section is configured to be positioned on a patient support apparatus of a magnetic resonance apparatus for performing the magnetic resonance examination. The bottom section and the top section have a total height in a connected state. The bottom section has a height of less than ⅓ of the total height.

This application claims the benefit of German Patent Application No. DE 10 2024 201 746.9, filed on Feb. 26, 2024, which is hereby incorporated by reference in its entirety.

### BACKGROUND

The present embodiments relate to a magnetic resonance local coil arrangement, a system with a magnetic resonance local coil arrangement and a patient support apparatus, a magnetic resonance apparatus, and a use of such apparatuses.

Imaging methods are an important tool of medical technology. High and variable soft tissue contrasts are one characteristic of imaging using magnetic resonance (MR), also referred to as magnetic resonance tomography (MRT) or more usually magnetic resonance imaging (MRI). A magnetic resonance apparatus is used here to generate a spatially homogeneous main magnetic field, in which a patient is located during a magnetic resonance examination. The patient is subjected to high frequency electromagnetic signals (RF signals) in order to excite the atomic nuclei of the patient. The excited atomic nuclei emit magnetic resonance signals that are received by one or more magnetic resonance local coil arrangements, local coils for short, and are then forwarded to an evaluation unit of the magnetic resonance apparatus. The evaluation unit uses the received magnetic resonance signals to calculate one or more magnetic resonance images.

When patients are examined in today's magnetic resonance apparatuses, patients who cannot walk by themselves are usually brought to a patient support apparatus of the magnetic resonance apparatus lying on another transport device (e.g., a movable bed or a couch). In the case of a fixed patient support apparatus, this may happen at the magnetic resonance apparatus or, in the case of mobile patient support apparatuses, at the location where the mobile patient support apparatuses are brought for positioning of the patient. Unwell and bed-bound patients, often also very heavy patients, require a lot of effort on the part of the radiology staff in order to transport the patients from one support surface to the other. Sheets or plastic mats are often laid under the patients, so that these may be gripped, and the patient may be transported from one support surface to the next by pulling this underlayer.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, positioning a patient on a patient support apparatus of a magnetic resonance apparatus may be made easier. Independent of the grammatical term usage, individuals with male, female, or other gender identities are included within the term.

A magnetic resonance local coil arrangement for receiving and/or sending RF signals is provided. The magnetic resonance local coil arrangement includes a bottom section and a top section, where, in a connected state, the bottom section and the top section delimit a spatial volume in which a body part of the patient may be supported during a magnetic resonance examination. The bottom section is configured to be positioned on a patient support apparatus of a magnetic resonance apparatus for performing the magnetic resonance examination. The bottom section and the top section have a total height in a connected state, where, in at least one section (e.g., a subsection) along the longitudinal axis of the patient, the bottom section has a height of less than ⅓ (e.g., less than ¼) of the total height.

The requirement for the bottom section to have a defined maximum height in at least one section along the longitudinal axis of the patient provides, for example, that the bottom section does not need to fulfill this requirement across the entire length along the longitudinal axis. The bottom section may also have a height in excess of this maximum height in another section along the longitudinal axis.

Due to the low height of the bottom section of only ⅓ (e.g., less than ¼) of the total height, the bottom section positioned on the patient support apparatus (e.g., when the patient is moved laterally onto the patient support apparatus) represents less of a barrier than conventional coils, as the bottom sections of these may have a height of around ½ of the total height. Thus, it is easier for a medical professional, for example, to move the patient onto the bottom section, as the height to be overcome is lower than is otherwise usual. This is an advantage, for example, if a patient is to be moved from a transport device (e.g., a movable bed or a movable couch) to a patient support apparatus of the magnetic resonance apparatus for the magnetic resonance examination of the body part. Such a body part may, for example, be the head of the patient, which then does not have to be lifted so far to be placed on the bottom section of the magnetic resonance local coil arrangement.

The longitudinal axis is, for example, the long axis of the patient. For example, the longitudinal axis runs from the feet of the patient to the head of the patient. When the patient is lying in a magnetic resonance apparatus, a longitudinal axis of the patient may be parallel to the z-axis of the magnetic resonance apparatus. The section along the longitudinal axis of the patient, in which the height is less than ⅓ (e.g., less than ¼) of the total height of the magnetic resonance local coil arrangement, may extend across a length of at least ⅓ (e.g., at least ½) of the total length of the magnetic resonance local coil arrangement along the longitudinal axis. In this section, the bottom section may represent a smaller hurdle when transporting the patient laterally. In one embodiment, the magnetic resonance local coil arrangement may only have a height of less than ⅓ (e.g., less than ¼) of the total height along its entire length along the longitudinal axis.

The bottom section of such a magnetic resonance local coil arrangement may also be referred to as the posterior section of a local coil; the top section may also be referred to as the anterior section. The connected state may be an operational state, in which RF signals (e.g., magnetic resonance signals) may be received and/or RF signals (e.g., excitation signals) may be sent.

The top section and/or the bottom section may have one or more antennas (e.g., coil elements) for receiving and/or sending RF signals. In a connected state, these antennas may be connected (e.g., electrically) to the magnetic resonance apparatus, so that received magnetic resonance signals may be transmitted to the magnetic resonance apparatus, for example.

The spatial volume is, for example, a three-dimensional spatial volume. In a connected state, the spatial volume may not be completely delimited by the bottom section and top section; for example, the delimitation of the spatial volume by the bottom section and the top section has a gap in at least one place (e.g., the magnetic resonance local coil arrangement has an opening in at least one place). During a magnetic resonance examination, the throat of the patient, for example, may be guided through such an opening while the head of the patient is located within the spatial volume.

The spatial volume may, for example, be delimited downward by a contact surface of the bottom section (e.g., in the direction of the force of gravity when the magnetic resonance local coil arrangement is used as intended). The height runs perpendicular to this contact surface. The contact surface may also be curved, so that the direction perpendicular to the contact surface may be location-dependent.

The direction of the height of the magnetic resonance local coil arrangement may be oriented in parallel (e.g., opposite) to the direction of the force of gravity when the magnetic resonance local coil arrangement is used as intended. The direction of the force of gravity may be labeled as the y-direction in the coordinate system of magnetic resonance apparatuses, while the z-direction usually indicates the direction of the main magnetic field in which the magnetic resonance imaging is taking place; in turn, the x-direction may be the direction orthogonal to the y- and z-direction. The force of gravity is ultimately also the force that is to be overcome when positioning a body part of the patient on the patient support apparatus.

The direction of the height of the magnetic resonance local coil arrangement is, for example, oriented perpendicular to the frontal plane of the patient, if the patient is lying on their back on the patient support apparatus.

The spatial volume may be delimited upward (e.g., against the direction of the force of gravity when the magnetic resonance local coil arrangement is used as intended) by the underside of the housing of the top section, for example (e.g., when the bottom section and top section are in the connected state). The spatial volume may be configured (e.g., is sufficiently large) to hold the body part of the patient that is to be examined. The body part may, for example, be the head, a foot, or a knee of the patient. In one embodiment, the body part may be placed onto the bottom section while the bottom section and top section are separated; the top section is then plugged into the bottom section to produce the connected state. The body part may be surrounded by antennas in the connected state in order to receive RF signals produced in the body part (e.g., magnetic resonance signals) and/or to send RF signals into the body part (e.g., excitation signals for the production of magnetic resonance signals).

The patient support apparatus includes, for example, a patient table on which the patient lies or is supported during the magnetic resonance examination. For example, the bottom section of a head coil is positioned at one end of the patient table for performing the magnetic resonance examination. The patient table may, for example, be movable on a fixed base frame of the patient support apparatus. This provides that the patient (e.g., the body part of the patient to be examined) may be moved into the main magnetic field, and thus, for example, into the isocenter of the magnetic resonance apparatus. This travel motion may be performed in the z-direction.

The total height of the bottom section and top section may be, for example, the sum of both sections. For example, if the bottom section and top section are interlocked in the connected state, the total height may also be smaller than the sum of the individual sections.

The magnetic resonance local coil arrangement may be configured to produce a mechanical and/or electrical connection between the bottom section and the top section when the top section is plugged into the bottom section. The mechanical and/or electrical connection may be produced directly and/or simultaneously when the bottom and top sections are plugged together. For example, the top section engages with the bottom section during the plugging process. In one embodiment, this provides that further movements are not required, and the workflow is simplified.

The top section may include at least one top plug connector, and the bottom section may include at least one bottom plug connector. The at least one top plug connector and the at least one bottom plug connector are configured to produce an electrical and/or mechanical connection between the top section and the bottom section when the top section is plugged into the bottom section.

The at least one top plug connector and the at least one bottom plug connector may be configured to correspond to each other in each case. For example, the at least one top plug connector includes one or more pin contacts that may engage with corresponding socket contacts on the at least one bottom plug connector. Electrical signals (e.g., RF signals) and/or electrical energy may be transmitted via the contacts.

The at least one bottom plug connector may be configured to be flush with the surface of the bottom section surrounding the at least one bottom plug connector. The at least one bottom plug connector may not protrude beyond the top edge of the bottom section. In one embodiment, the body part of the patient may be positioned in the bottom section with particular safety and ease, because the at least one bottom plug connector has no obstructive contours (e.g., ones pointing upward).

The bottom section may have a cover that is suitable (e.g., to cover any electrical contacts of the at least one bottom plug connector) to be positioned (e.g., pushed) over the at least one bottom plug connector. This may increase the safety of the patient and/or the operating personnel.

One possible embodiment variant provides for the magnetic resonance local coil arrangement to be or include a head coil, a knee coil, or a foot coil (e.g., a foot/ankle coil). Accordingly, the body part to be examined may be the head, a knee, or a foot of the patient. These body parts may be placed in the bottom section of each coil with particular ease.

One possible embodiment variant provides for the bottom section to be part of a spine coil (e.g., a very long spine coil). For example, the magnetic resonance local coil arrangement is a combination of a spine coil with another local coil having the bottom section.

One possible embodiment variant provides for the magnetic resonance local coil arrangement as the top section to include a first top section for examination of a first body part of the patient, where the top section may be connected to the bottom section for examination of the first body part. The magnetic resonance local coil arrangement also includes a second top section for examination of a second body part of the patient. The second top section may be connected to the bottom section for examination of the second body part. Both the first top section and the second top section may be connected to the same bottom section (e.g., may form a connected state).

The magnetic resonance local coil arrangement may thus, for example, be an arrangement in which the bottom section may be combined with various top sections that are each configured for examination of a different body part. The bottom section of the magnetic resonance local coil arrangement may be used multiple times for different applications.

For example, the first body part is the head of the patient, and the second body part is the ankle/foot area. Accordingly, the first top section is a top section that is configured, together with the bottom section, to examine the head of the patient, and the second top section is a top section that is configured, together with the bottom section, to examine the ankle/foot area of the patient.

Further, a system is provided, including a magnetic resonance local coil arrangement for receiving and/or sending RF signals and a patient support apparatus for performing the magnetic resonance examination with a magnetic resonance apparatus. The magnetic resonance local coil arrangement is, for example, a magnetic resonance local coil arrangement described above.

The patient support apparatus may have a table surface on which a lying patient is intended to be supported during the performance of a magnetic resonance examination (e.g., magnetic resonance measurement). The bottom section protrudes less than 5 cm above the table surface in at least one section along the longitudinal axis of the patient when the bottom section of the magnetic resonance local coil arrangement is mounted on the patient support apparatus. For example, the form of the table surface may predominantly be described as a plane, and the bottom section protrudes less than 5 cm above this plane in at least one section along the longitudinal axis of the patient. The section along the longitudinal axis of the patient, in which the bottom section protrudes less than 5 cm above the table surface when the bottom section of the magnetic resonance local coil arrangement is mounted on the patient support apparatus, may extend across a length of at least ⅓ (e.g., at least ½) of the total length of the magnetic resonance local coil arrangement along the longitudinal axis. In one embodiment, this enables the operating personnel to position the patient on the table surface with particular ease.

The patient support apparatus may, for example, include a patient table with the table surface and a support (e.g., a frame), on which the patient table is mounted. In one embodiment, the patient table is movable, so that the body part of the patient may be brought to the optimal position in the magnetic resonance apparatus.

The bottom section may have an interface to the top section, where this interface is flush with the table surface when the bottom section of the magnetic resonance local coil arrangement is mounted on the patient support apparatus. The bottom section of the magnetic resonance local coil arrangement may not protrude beyond the table surface, so that the patient may be moved with particular ease on the patient support apparatus or may move themselves with particular ease on the patient support apparatus.

One possible embodiment variant provides for the patient support apparatus to include at least one table plug connector table and for the top section of the magnetic resonance local coil arrangement to include at least one coil plug connector. The at least one table plug connector and the at least one coil plug connector are configured to produce an electrical connection between the patient support apparatus and the top section of the magnetic resonance local coil arrangement when the at least one coil plug connector is connected to the at least one table plug connector. The top section of the magnetic resonance local coil arrangement may include, for example, at least one cable, connecting the at least one coil plug connector to the rest of the top section.

The bottom section of the magnetic resonance local coil arrangement may have at least one electrical contact to provide electrical contacting of the top section of the magnetic resonance local coil arrangement. The at least one electrical contact is electrically decoupled.

Another embodiment variant of the system provides for the bottom section of the magnetic resonance local coil arrangement to be permanently integrated in the patient support apparatus. This may be the bottom section of a foot/ankle coil.

Further, a magnetic resonance apparatus is provided that includes a system described above of magnetic resonance local coil arrangement and patient support apparatus.

Use of a magnetic resonance apparatus described above and/or a system described above of magnetic resonance local coil arrangement and patient support apparatus and/or a magnetic resonance apparatus described above is also provided for performing a magnetic resonance examination.

Further advantages, features, and details of the present embodiments are specified in the example embodiments described below and in the drawings. Correlating parts have been given the same reference numbers in all figures.

### BRIEF DESCRIPTION OF THE DRAWINGS

FIG. 1 shows a magnetic resonance apparatus with a patient support apparatus and a magnetic resonance local coil arrangement;

FIG. 2 shows a magnetic resonance local coil arrangement according to the prior art;

FIG. 3 shows a magnetic resonance local coil arrangement with a low bottom section;

FIG. 4 shows a patient on a transport device before a transfer to a patient support apparatus;

FIG. 5 shows a patient after a transfer to a patient support apparatus;

FIG. 6 shows a patient on a patient support apparatus with plugged in top section;

FIG. 7 shows a system of magnetic resonance local coil arrangement and patient support apparatus;

FIG. 8 shows the underside of a top section of a magnetic resonance local coil arrangement;

FIG. 9 shows a magnetic resonance local coil arrangement with a bottom section and two alternative top sections; and

FIG. 10 shows a magnetic resonance local coil arrangement with a bottom section that has a height of less than ⅓ of a total height in only one partial section.

### DETAILED DESCRIPTION

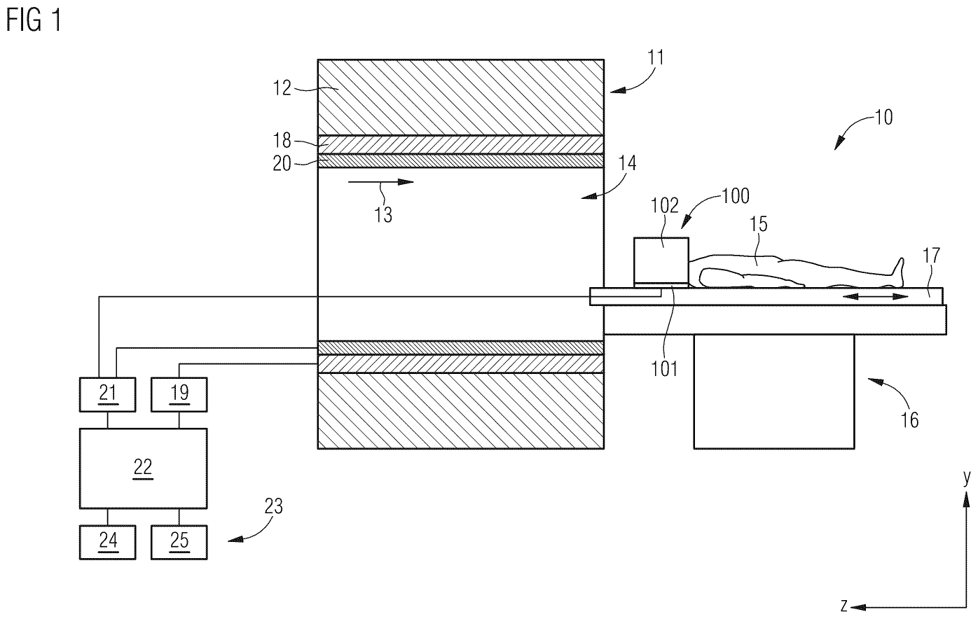

FIG. 1 shows a schematic representation of a magnetic resonance apparatus 10. The magnetic resonance apparatus 10 includes a magnetic unit 11 that has a main magnet 12 to generate a strong and, for example, temporally constant main magnetic field 13. The magnetic resonance apparatus 10 also includes a patient receiving area 14 for receiving a patient 15. The patient receiving area 14 in the present example embodiment is cylindrical in design and is enclosed in a circumferential direction by the cylindrical magnetic unit 11.

However, a deviating embodiment of the patient receiving area 14 may be provided at any time. The patient 15 may be moved into the patient receiving area 14 by a patient support apparatus 16 of the magnetic resonance apparatus 10. The patient support apparatus 16 has a patient table 17 within the patient receiving area 14 for this purpose that is configured to move in the z-direction. The z-direction equates to the longitudinal axis or long axis of the patient 15. The z-direction also represents the central axis of the cylindrical magnetic unit 11.

The magnetic unit 11 also has a gradient coil unit 18 for generating magnetic field gradients that are used for spatial encoding during imaging. The gradient coil unit 18 is controlled by a gradient control unit 19 of the magnetic resonance apparatus 10. The magnetic unit 11 also includes a radio frequency antenna unit 20 that in the present example embodiment includes a body coil permanently integrated in the magnetic resonance apparatus 10. The radio frequency antenna unit 20 is controlled by a radio frequency antenna control unit 21 of the magnetic resonance apparatus 10 and emits radio frequency magnetic resonance sequences into an examination area that is essentially formed by a patient receiving area 14 of the magnetic resonance apparatus 10. This results in the atomic nuclei being excited by the main magnetic field 13 generated by the main magnet 12. Magnetic resonance signals are produced by relaxing the excited atomic nuclei. The body part of the patient being examined in this example is the head. For this purpose, a magnetic resonance local coil arrangement 100 in the form of a head coil is positioned at the head of the patient 15, which is used to receive the magnetic resonance signals. The magnetic resonance local coil arrangement 100 includes multiple antennas for this purpose, in which magnetic resonance signals induce excitement that is measured. The magnetic resonance local coil arrangement 100 includes a bottom section 101 and a top section 102 that may be plugged into the bottom section; this creates a spatial volume between the bottom section 101 and a top section 102, in which the head is placed.

The magnetic resonance apparatus 10 has a system control unit 22 to control the main magnet 12, the gradient control unit 19, and the radio frequency antenna control unit 21. The system control unit 22 controls the magnetic resonance apparatus 10 centrally, by performing a predefined imaging gradient echo sequence, for example. The system control unit 22 also includes an evaluation unit that is not shown in detail, for evaluation of the magnetic resonance signals that are measured during the magnetic resonance examination. The magnetic resonance apparatus 10 also includes a user interface 23 that is connected to the system control unit 22. Control information, such as imaging parameters and reconstructed magnetic resonance images, may be displayed for medical operating personnel on a display unit 24, on at least one monitor, for example, of the user interface 23. The user interface 23 also has an input unit 25 that medical operating personnel may use to enter information and/or parameters during a measurement procedure.

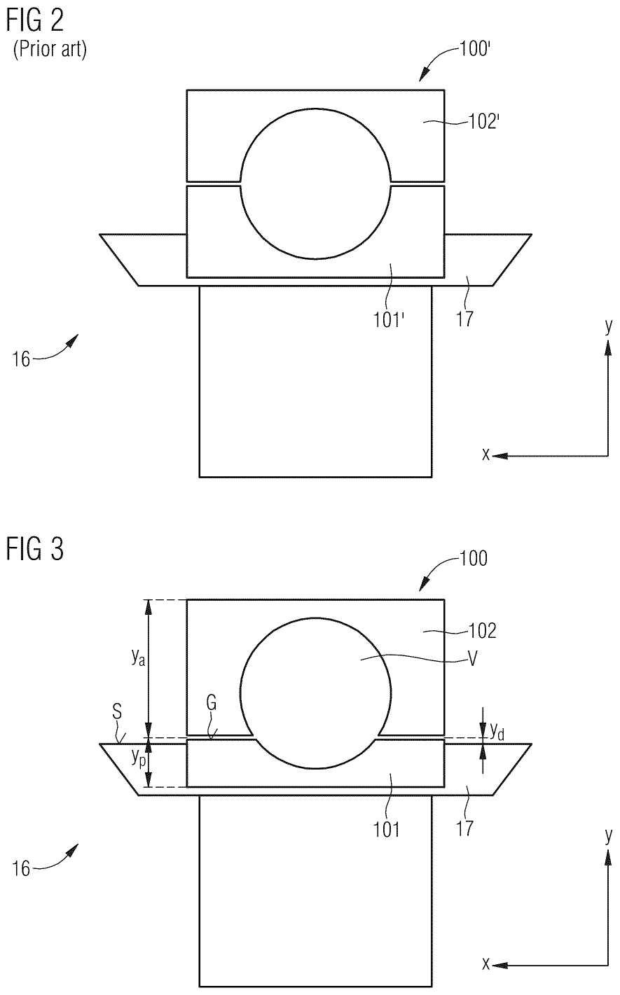

FIG. 2 shows a conventional magnetic resonance local coil arrangement 100′ (e.g., a head coil), on which the bottom section 101′ is effectively the same height as the top section 102′.

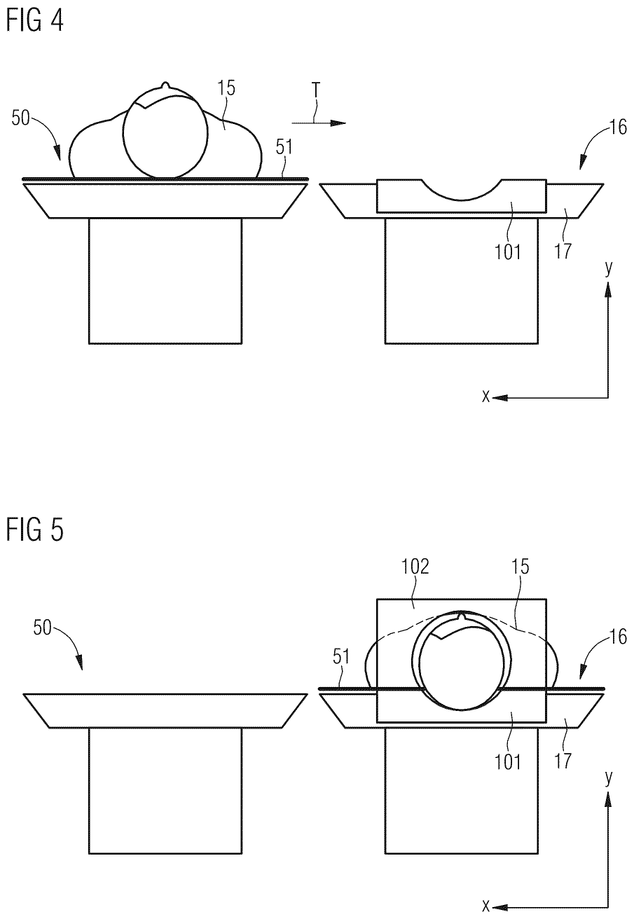

The geometry of the patient table 17 together with the magnetic resonance local coil arrangement 100′ represents a problem here. In the usual positioning (e.g., head first), the head of the patient 15 may be placed in the bottom section 101′ of the head coil. In head examinations, this may serve as the receiving coil, and in non-head examinations, simply as an underlayer or support for the head. In the case of lateral transport T of the patient 15 (e.g., perpendicular to their longitudinal axis), as shown in the example in FIGS. 3-4, from a couch 50 on the patient table 17, the bottom section of the head coil is a hindrance, however; the contact surfaces of the couch 50 and the patient table 17 are predominantly flat and allow for easy lateral transport T of the body, but the bottom section 101′ of the head coil projects many centimeters above the surface of the patient table 17. This makes the workflow of the magnetic resonance examination difficult. The head of the patient 15 is to be lifted over the edges of the head coil, or the patient 15 is to be transported further laterally at their feet and then pushed into the head coil in the z-direction along their longitudinal axis.

Therefore, a magnetic resonance local coil arrangement 100 for receiving and/or sending RF signals is provided, as schematically represented in FIG. 3. The magnetic resonance local coil arrangement 100 is a head coil in this example, but the present embodiments may also be transferred to other local coils in multiple parts, such as knee coils or foot coils (e.g., foot/ankle coils).

The magnetic resonance local coil arrangement 100 includes a bottom section 101 and a top section 102, where, in a connected state, the bottom section 101 and the top section 102 delimit a spatial volume V in which a body part of a patient (e.g., the head) may be supported during a magnetic resonance examination. The bottom section 101 may be positioned on a patient table 17 of a patient support apparatus 16 of a magnetic resonance apparatus 10 for performing the magnetic resonance examination. The bottom section 101 has a height of yₚ, and the top section 102 has a height of yₐ. In a connected state, the bottom section 101 and the top section 102 have a total height of y_{g}=yₚ+yₐ. The bottom section 101 has a height of less than ⅓ (e.g., less than ¼) of the total height y_{g}. The low height of the bottom section 101 makes it easier to lift the patient 15 over the edges of the bottom section 101.

The height of one section is the dimension (e.g., the reach) of the section in the y-direction (e.g., parallel to the force of gravity when used as intended during a magnetic resonance examination).

The patient table 17 of the patient support apparatus 16 has a table surface S on which the patient 15 may be supported during a performance of the magnetic resonance examination. The table surface S is mainly level. The patient table 17 has a depression in the y-direction in which the magnetic resonance local coil arrangement 100 (e.g., the bottom section 101) may be arranged.

The dimension y_{d}, by which the bottom section 101 protrudes upward above the table surface S (e.g., in the y-direction) may be less than 5 cm when the bottom section 101 of the magnetic resonance local coil arrangement 100 is mounted on the patient support apparatus 16. y_{d} may be equal to zero (e.g., the bottom section 101 has an interface G to the top section 102, where this interface G is flush with the table surface S when the bottom section 101 of the magnetic resonance local coil arrangement 100 is mounted on the patient support apparatus 16).

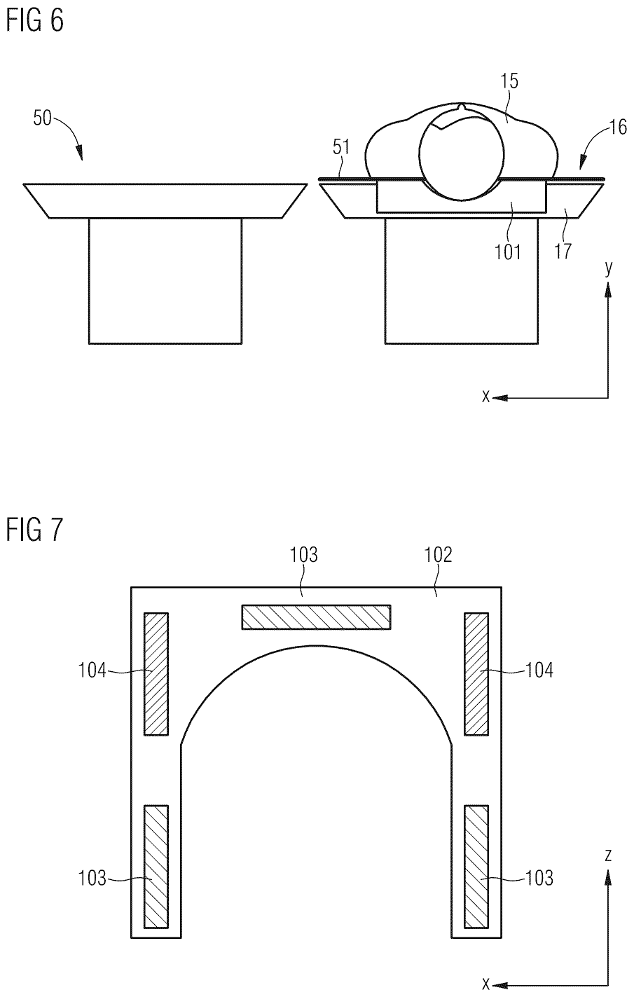

In FIGS. 4-6, the use of the magnetic resonance local coil arrangement 100 on the patient support apparatus 16 is represented as part of a magnetic resonance examination. The patient 15 is brought alongside the patient support apparatus 16 on a transport device in the form of a couch 50. The patient 15 is then lifted in the horizontal x-direction onto the patient support apparatus 16 (e.g., a lateral transport T is performed). A underlayer 51 (e.g., a sheet or plastic mat) may be used, so that this may be gripped, and the patient 15 may be transported onto this patient table 17 by pulling this underlayer 51. If the head is then positioned on the bottom section 101 and the rest of the body of the patient 15 is lying on the table surface S, the top section 102 may be brought down from above onto the bottom section 101 (e.g., may be plugged into the bottom section 101).

A mechanical and/or electrical connection between the bottom section 101 and the top section 102 may be produced when the top section 102 is plugged into the bottom section 101. For example, the top section 102 includes one or more top plug connectors, and the bottom section 101 includes one or more corresponding plug connectors that are suitable for producing an electrical and/or mechanical connection between the top section 102 and the bottom section 101 when the top section 102 is plugged into the bottom section 101. An example of this is illustrated in FIG. 7, which shows a view of the underside of the top section. Multiple top plug connectors are shown on the underside in the form of multiple mechanical connecting elements 103 and multiple electrical connecting elements 104, which interact with corresponding connecting elements on the upper side of the bottom section (not shown here) when being plugged in. Any magnetic resonance signals received by the top section 102 of the magnetic resonance local coil arrangement 100 may then be transmitted via the electrical connecting elements 104 to the bottom section of the magnetic resonance local coil arrangement 100.

The bottom plug connectors may be configured to be flush with the surface of the interface G of the bottom section 101 surrounding the at least one bottom plug connector, so that they do not interfere with the transport T. The bottom section 101 may include one or more covers that are suitable to be positioned over the bottom plug connectors (e.g., to cover any electrical contacts of the bottom plug connectors).

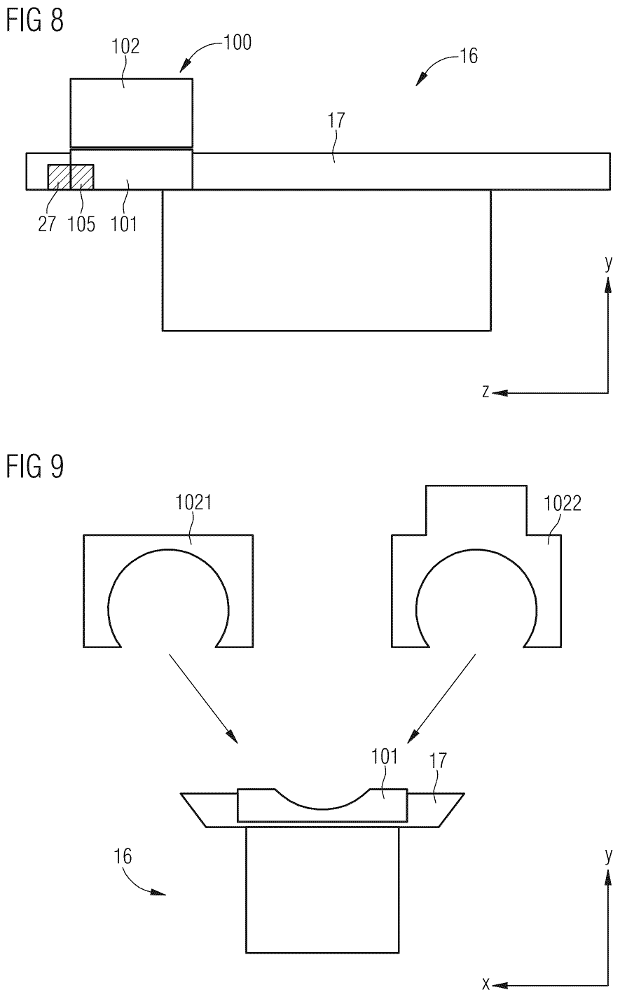

FIG. 8 shows a patient support apparatus 16 with a table plug connector 27. The bottom section 101 of the magnetic resonance local coil arrangement 100 includes a coil plug connector 103. The table plug connector 27 and the coil plug connector 105 produce an electrical connection between the patient support apparatus 16 and the top section of the magnetic resonance local coil arrangement 100 when the bottom section 101 is positioned on the patient table. Any magnetic resonance signals received by the magnetic resonance local coil arrangement 100 may then be transmitted via this interface, for example, to the system control unit 22 of the magnetic resonance apparatus 10.

In one embodiment, the top section 102 of the magnetic resonance local coil arrangement 100 includes at least one coil plug connector that may be plugged into a table plug connector to produce an electrical connection between the patient support apparatus 16 and the top section 102 of the magnetic resonance local coil arrangement 100, so that any magnetic resonance signals received by the top section 102 may be transmitted directly to the patient support apparatus 16.

In one embodiment, the bottom section 101 is permanently integrated in the patient support apparatus 16 (e.g., in the patient table 17). Further, the bottom section 101 may be part of a spinal coil (e.g., a very long spinal coil).

FIG. 9 illustrates that various top sections 1021, 1022 may be used with the same bottom section 101. For example, the magnetic resonance local coil arrangement 100 may include, as the top section, a first top section 1021 for examining the head of the patient 15, where the first top section 1021 may be connected to the bottom section 101 for examination of the head; further, the magnetic resonance local coil arrangement 100 may include a second top section 1022 for examination of an ankle/foot area of the patient 15, where the second top section 1022 may be connected to the bottom section 101 for examination of the ankle/foot area. Multiple usage of the bottom section 101 like this may reduce costs.

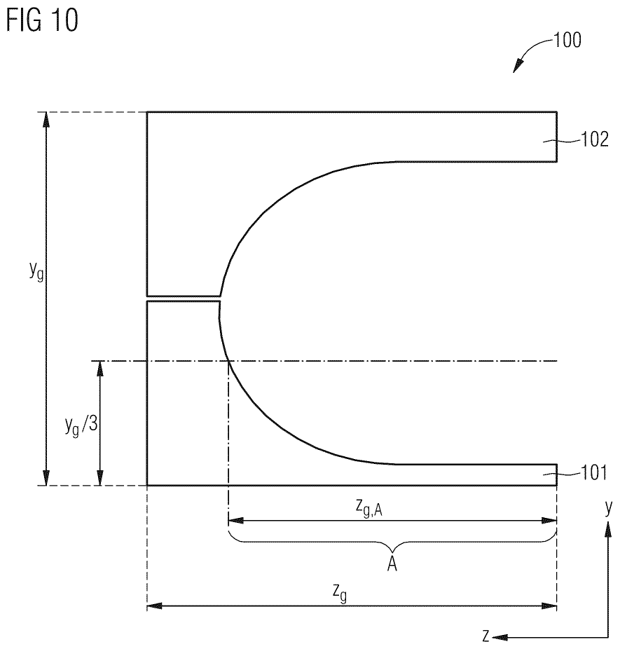

FIG. 10 shows an example of a magnetic resonance local coil arrangement 100, in which the bottom section only has a height of ⅓ of the total height y_{g} in one section A along the z-direction (e.g., along the longitudinal axis of the patient 15 when used as intended). While the height of the bottom section 101 at the head end (e.g., left in FIG. 10) is around half the total height y_{g}, the height always reduces in the negative z-direction until the height drops below the height of ⅓ of the total height y_{g}. In section A, the head of the patient may be lifted laterally with particular ease in the bottom section 101 of the magnetic resonance local coil arrangement 100. The length Z_{g,A} of the section A, in which the height falls below ⅓ of the total height y_{g}, is more than ½ of the total length z_{g} here of the magnetic resonance local coil arrangement along the longitudinal axis. In one embodiment, no or very little movement in the z-direction is required after the lateral transport, in order to bring the head of the patient to its end position at which the magnetic resonance measurement is performed.

The apparatuses described in detail above merely concern example embodiments that may be modified by a person skilled in the art in a wide variety of ways without departing from the protective scope of the invention. Further, use of the indefinite article “a” or “an” does not prevent the features concerned being present multiple times. Likewise, terms such as “unit” do not exclude the possibility that the components concerned include multiple interacting subcomponents that may be distributed, including spatially if applicable.

The elements and features recited in the appended claims may be combined in different ways to produce new claims that likewise fall within the scope of the present embodiments. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A magnetic resonance local coil arrangement for receiving, sending, or receiving and sending radio frequency (RF) signals, the magnetic resonance local coil arrangement comprising:
a bottom section and a top section,
wherein, in a connected state, the bottom section and the top section delimit a spatial volume in which a body part of a patient is positionable during a magnetic resonance examination,
wherein the bottom section is configured to be positioned on a patient support apparatus of a magnetic resonance apparatus for performing the magnetic resonance examination,
wherein, in a connected state, the bottom section and the top section have a total height, and
wherein, in at least one section along a longitudinal axis of the patient, the bottom section has a height of less than ⅓ of the total height.

2. The magnetic resonance local coil arrangement of claim 1, wherein, in the at least one section along the longitudinal axis of the patient, the bottom section has the height of less than ¼ of the total height.

3. The magnetic resonance local coil arrangement of claim 1, wherein the bottom section has a contact surface to support the body part that delimits the spatial volume downward, and
wherein a direction of the height of the magnetic resonance local coil arrangement is oriented in parallel to the force of gravity when the magnetic resonance local coil arrangement is positioned on the patient support apparatus of the magnetic resonance apparatus for performing a magnetic resonance examination.

4. The magnetic resonance local coil arrangement of claim 1, wherein the magnetic resonance local coil arrangement is configured to produce a mechanical, electrical, or mechanical and electrical connection between the bottom section and the top section when the top section is plugged into the bottom section.

5. The magnetic resonance local coil arrangement of claim 1, wherein the top section comprises at least one top plug connector,
wherein the bottom section comprises at least one bottom plug connector, and
wherein the at least one top plug connector and the at least one bottom plug connector are configured to produce an electrical, mechanical, or electrical and mechanical connection between the top section and the bottom section when the top section is plugged into the bottom section.

6. The magnetic resonance local coil arrangement of claim 5, wherein the at least one bottom plug connector is configured to be flush with the contact surface of the bottom section surrounding the at least one bottom plug connector.

7. The magnetic resonance local coil arrangement of claim 5, wherein the bottom section has a cover that is suitable to be positioned over the at least one bottom plug connector.

8. The magnetic resonance local coil arrangement of claim 7, wherein cover is for covering any electrical contacts of the at least one bottom plug connector.

9. The magnetic resonance local coil arrangement of claim 1, wherein the magnetic resonance local coil arrangement is or comprises a head coil, a knee coil, or a foot coil.

10. The magnetic resonance local coil arrangement of claim 1, wherein the magnetic resonance local coil arrangement is or comprises a foot/ankle coil.

11. The magnetic resonance local coil arrangement of claim 1, wherein the bottom section is part of a spine coil.

12. The magnetic resonance local coil arrangement of claim 1, wherein the magnetic resonance local coil arrangement as the top section comprises a first top section for examination of a first body part of the patient,
wherein the top section is connectable to the bottom section for examination of the first body part,
wherein the magnetic resonance local coil arrangement comprises a second top section for examination of a second body part of the patient, and
wherein the second top section is connectable to the bottom section for examination of the second body part.

13. A system comprising:
a magnetic resonance local coil arrangement for receiving, sending, or receiving and sending radio frequency (RF) signals; and
a patient support apparatus for performing a magnetic resonance examination with a magnetic resonance apparatus,
wherein the patient support apparatus has a table surface that is intended to support a patient while the magnetic resonance examination is performed,
wherein a bottom section of the magnetic resonance local coil arrangement protrudes less than 5 cm above the table surface in at least one section along a longitudinal axis of the patient when the bottom section of the magnetic resonance local coil arrangement is mounted on the patient support apparatus.

14. The system as claimed in claim 13, wherein the bottom section has an interface to a top section of the magnetic resonance local coil arrangement, and
wherein the interface is flush with the table surface when the bottom section of the magnetic resonance local coil arrangement is mounted on the patient support apparatus.

15. The system of claim 13, wherein the patient support apparatus comprises at least one table plug connector,
wherein the top section of the magnetic resonance local coil arrangement comprises at least one coil plug connector, and
wherein the at least one table plug connector and the at least one coil plug connector are configured to produce an electrical connection between the patient support apparatus and the top section of the magnetic resonance local coil arrangement when the at least one coil plug connector is connected to the at least one table plug connector.

16. The system of claim 13, wherein the bottom section is permanently integrated in the patient support apparatus.

17. A magnetic resonance apparatus comprising:
a magnetic resonance local coil arrangement for receiving, sending, or receiving and sending radio frequency (RF) signals, the magnetic resonance local coil arrangement comprising:
a bottom section and a top section, wherein, in a connected state, the bottom section and the top section delimit a spatial volume in which a body part of a patient is positionable during a magnetic resonance examination, wherein the bottom section is configured to be positioned on a patient support apparatus of a magnetic resonance apparatus for performing the magnetic resonance examination, wherein, in a connected state, the bottom section and the top section have a total height, and wherein, in at least one section along a longitudinal axis of the patient, the bottom section has a height of less than ⅓ of the total height; or
a system comprising:
the magnetic resonance local coil arrangement; and
the patient support apparatus for performing the magnetic resonance examination with the magnetic resonance apparatus, wherein the patient support apparatus has a table surface that is intended to support the patient while the magnetic resonance examination is performed, wherein the bottom section of the magnetic resonance local coil arrangement protrudes less than 5 cm above the table surface in at least one section along the longitudinal axis of the patient when the bottom section of the magnetic resonance local coil arrangement is mounted on the patient support apparatus.

18. A method comprising:
performing a magnetic resonance examination using a magnetic resonance local coil arrangement, a system, or the magnetic resonance local coil arrangement and the system,
wherein the magnetic resonance local coil arrangement is for receiving, sending, or receiving and sending radio frequency (RF) signals and comprises:
a bottom section and a top section, wherein, in a connected state, the bottom section and the top section delimit a spatial volume in which a body part of a patient is positionable during a magnetic resonance examination, wherein the bottom section is configured to be positioned on a patient support apparatus of a magnetic resonance apparatus for performing the magnetic resonance examination, wherein, in a connected state, the bottom section and the top section have a total height, and wherein, in at least one section along a longitudinal axis of the patient, the bottom section has a height of less than ⅓ of the total height, and
wherein the system comprises:
the magnetic resonance local coil arrangement; and
the patient support apparatus for performing the magnetic resonance examination with the magnetic resonance apparatus, wherein the patient support apparatus has a table surface that is intended to support the patient while the magnetic resonance examination is performed, wherein the bottom section of the magnetic resonance local coil arrangement protrudes less than 5 cm above the table surface in at least one section along the longitudinal axis of the patient when the bottom section of the magnetic resonance local coil arrangement is mounted on the patient support apparatus.
